(19) United States
(12) Patent Application Publication
(10) Pub. No.: US 2026/0308718 A1
(43) Pub. Date: Oct. 8, 2026

(54) METHOD FOR PREPARING A METAL ALLOY

(71) Applicants: CONSTELLIUM NEUF-BRISACH, BIESHEIM (FR); CONSTELLIUM MUSCLE SHOALS LLC, MUSCLE SHOALS, AL (US)

(72) Inventors: Georges SALLOUM ABOU JAOUDE, GRENOBLE (FR); Timothy WARNER, CORENC (FR); Pierre-Yves MENET, VOREPPE (FR)

(73) Assignees: CONSTELLIUM NEUF-BRISACH, BIESHEIM (FR); CONSTELLIUM MUSCLE SHOALS LLC, MUSCLE SHOALS, AL (US)

(21) Appl. No.: 19/481,582

(22) PCT Filed: Apr. 24, 2024
(86) PCT No.: PCT/FR2024/050537

§ 371 Date: Nov. 5, 2025

(30) Foreign Application Priority Data
May 5, 2023 (FR) FR2304531

(51) Int. Cl. C22C 1/02; C22B 7/00; C22B 21/00; C22C 1/03; C22C 21/02

(52) U.S. Cl. CPC C22C 1/026; C22B 7/003; C22B 21/0092; C22C 1/03; C22C 21/02

(57) ABSTRACT

The invention relates to a method for preparing a liquid metal bath of a metal alloy having a chemical composition comprising at least one first element (X1) and at least one second element (X2), the preparation method comprising a charging step (b) in which all or part of the variable metal waste is continuously introduced into a melting furnace, and is melted to obtain all or part of a liquid metal bath, and a measuring step (c) wherein a composition of said liquid metal bath is continuously measured using a laser-induced plasma atomic emission spectrometry device (3) also called LIBS device, so as to continuously measure at least one content of said first element (X1) and a content of said second element (X2) in the liquid metal bath.

### TECHNICAL FIELD OF THE INVENTION

The present invention relates to a method for preparing a metal alloy allowing the use of metal waste. The method is particularly interesting for manufacturing an aluminum alloy having a final composition comprising at least one chemical element of interest different from aluminum from aluminum waste.

### PRIOR ART

In the field of metallurgy, and in particular in the manufacture of aluminum alloys, preparing aluminum alloys by melting a primary aluminum obtained directly from bauxite to obtain a relatively pure liquid aluminum bath is known. In order to then prepare an alloy of precise composition, additives of chemical elements expected in the final composition are added to the previously obtained pure aluminum bath.

Once all the required additives have been added, provision is made to take a small amount of the liquid metal bath to form a test piece commonly called a “slug”, which is machined and prepared for laboratory analysis, in order to determine whether the composition of the liquid metal bath is close to the expected composition for the aluminum alloy. If the composition is not close to what is expected, then an adjustment of the composition can be made by adding new additives to the liquid metal bath. Sometimes, when the composition is too far away from the expected composition, it happens that the liquid metal bath is discharged from the furnace, to start a new preparation process.

Although this method is satisfactory in that it makes it possible to prepare a bath of liquid aluminum alloy to a given composition, it first requires the use of pure materials, or of known composition. Moreover, it is always necessary to wait for the slug analysis to be done to continue the preparation process, which involves a loss of time, and therefore of energy, to keep the liquid metal at temperature in the melting furnace.

An economically and environmentally advantageous solution with regard to the supply of the initial materials is to use metal waste and, in the case of aluminum, aluminum waste. This is because the cost for purchasing aluminum waste is lower than the production cost of pure aluminum, and reduces the manufacturing CO₂ footprint, and the reuse of waste enables aluminum waste to be disposed of.

There are various sources of supply for metal waste that can be used for recycling. Depending on the source of supply, the composition of metal waste may be more or less known. It is also possible to classify the sources of supply between so-called controlled sources and so-called mixed sources.

The controlled sources include aluminum waste of relatively known composition. For example, they correspond to waste from the processing processes implemented by manufacturers of aluminum alloys, such as plate feet or heads that are removed at the end of a casting process or cutting waste during the rolling steps. Controlled sources may also comprise waste produced by manufacturers of products made from aluminum (or “pre-consumer” scrap according to the dedicated Anglo-Saxon terminology). The particularity of all this waste from controlled sources is that it has a composition that corresponds overall to the composition of the alloy that has been cast.

Mixed sources include aluminum waste with a more disparate composition. This is because this aluminum waste can, for example, correspond to aluminum waste that has been recovered following its use by the end user. In particular, it may correspond, for example, to waste from beverage containers also known as UBC (Used Beverage Can according to the dedicated Anglo-Saxon terminology) or aluminum waste from car grinding. Alternatively, this aluminum waste may correspond to waste of known composition but mixed with other waste of a different type, so-called mixed metal waste. In particular, this may include mixed “pre-consumer” waste, or waste from a casting process that has been mixed. The particularity of all this mixed metal waste is that its composition is not constant depending on the source of supply. It can vary not only because it can come from different sources but also because there may be mixtures therein. This disparity in composition leads to difficulties in using this waste in alloy-production processes.

The ISRI, standing for “Institute of Scrap Recycling Industries” (www.ISRI.org), offers technical definitions of metal waste in its ISRI Scrap Specifications Circular. In particular, the section Guidelines for Nonferrous Scrap: NF-2022 (edition 07/2022), which can be consulted on the website http://www.scrap2.org/specs/, for example presents various types of variable aluminum waste. For example, the circular presents variable aluminum waste called “Zorba” defined as crushed non-ferrous waste, and which is mainly made of aluminum. However, copper, lead, magnesium, stainless steel, nickel, tin and zinc can be found in the Zorba in elemental or alloyed (solid) form. Waste of the “Zorba” type can be better defined when its name is followed by a number, such as Zorba 90 which means that the material contains about 90% non-ferrous metals. Zorba is the result of a typical, but not limited, treatment of automotive shredder residues (ASR) by known methods: first by ferrous separation with the aim of reducing or eliminating iron and/or large iron fixings; after creating a mixed fraction of waste and non-ferrous metals with minimal iron content, the fraction can be separated in a non-ferrous metal separator, such as an eddy current. An eddy current separator separates the majority of non-ferrous metals from the other components. The result of this separation is Zorba waste, a product of mixed non-ferrous metals.

Aluminum and magnesium are two specific components of Zorba produced by further processing the virgin ASRs to separate the components thereof. Both aluminum and magnesium have commercial value—a value that increases if these metals are extracted from Zorba waste. One of these products of commercial value is “Twitch”, which is an aluminum product derived from wet or dry separation of ASR, as defined by the Institute of Scrap Recycling Industries, Inc.

Twitch waste can be obtained from an automobile, at the end of its useful life after it has been shredded. To be classified as “Twitch”, the material must be dry and not contain more than a maximum of 1% free zinc, a maximum of 1% free magnesium and a maximum of 1% analytical iron. In addition, the material must not contain more than 2% in total non-metallic materials, including a maximum of 1% rubber and plastic.

The use of mixed metal waste, especially the use of mixed aluminum waste, can pose a number of problems if it are reused in a preparation process. This is because, when preparing a liquid aluminum bath to prepare an aluminum alloy belonging to a given series, it is necessary to maintain the content of certain given chemical elements in a particular content range. For example, it may be necessary to guarantee at least the presence of a first chemical element in a given content range, defined by a non-zero minimum value and a maximum value, and to limit to a maximum the presence of a second chemical element. If the content of said first chemical element in the variable aluminum waste is too low, and/or if the content of said second chemical element in the variable aluminum waste is too high relative to the given value range, it will be very difficult or even impossible to prepare an aluminum alloy corresponding to the targeted composition. Moreover, even if the first and second chemical element contents of the waste are suitable for the manufacture of the aluminum alloy, it is not easy to know exactly the composition of the alloy formed without using a slug sample.

U.S. Pat. No. 6,784,429 discloses a method and equipment and an in-situ method for measuring in real time the properties of liquids, and in particular of liquid metal.

A report commissioned by the U.S. Department of Energy “In Situ, Real Time Measurement of Melt Constituents in the Aluminum, Glass and Steel Industries” written by Robert De Saro et al. discloses the use of LIBS on an industrial scale.

In order to be able to use waste with a variable composition, sorting the waste according to its chemical composition in order to reduce uncertainty as to the composition is known. In particular, the document US20180297091 discloses a method of analysis on a solid of aluminum waste mixtures so as to determine the composition of the mixture so as to be able to use it during the alloy-production step. The aluminum waste is analyzed before melting.

U.S. Pat. No. 9,956,609B1 discloses a method for using aluminum waste that combines a analysis method on solids of aluminum waste mixtures so as to determine the composition of the mixture associated with a continuous analysis of the liquid metal bath by a laser-induced plasma atomic emission spectrometry method also called “LIBS” for Laser Induced Breakdown Spectroscopy according to the dedicated English terminology. Although these methods make it possible to prepare aluminum alloys from waste, they require either the use of waste with a known composition, or the implementation of an upstream analysis step that eliminates a large part of the mixed waste, and increases the production time of the aluminum alloy, which has obvious repercussions on the total cost of the manufacturing process.

Thus, there is a need to find a method that makes it possible to both use a maximum of mixed metal waste to prepare an aluminum alloy of a given composition, without performing a specific selection of the mixed metal waste prior to the preparation of the liquid metal bath, and by limiting the total time of implementation of the method to limit energy losses, and thus reduce the environmental and financial cost of the manufacturing method.

### DISCLOSURE OF THE INVENTION

The present invention aims to propose a solution that addresses all or some of the aforementioned problems.

This aim can be achieved by implementing a method for preparing a liquid metal bath of a metal alloy having a chemical composition comprising at least one first element selected from Cu, Mg, Si, Zn, Li, Mn, Zr, Cr, Fe, Ti, V, Ca, Be, Sc, Cd, Pb, Na and Ni, present in said chemical composition at a first content c1 lying between a first lower target threshold X1ₘᵢₙ, and a first target upper threshold X1ₘₐₓ; and at least one second element selected from Cu, Mg, Si, Zn, Li, Mn, Zr, Cr, Fe, Ti, V, Ca, Be, Sc, Cd, Pb, Na and Ni, present in said chemical composition at a second content c2, less than a second target upper threshold X2ₘₐₓ, the preparation method comprising:
a) a provisioning step in which an amount m1 of variable metal waste is provided, said variable metal waste being characterized by a mean charge content w1 in said at least one first element lying between a first lower initial threshold Y1ₘᵢₙ and a second upper initial threshold Y1ₘₐₓ, and such that Y1ₘₐₓ>X1ₘₐₓ and (Y1ₘₐₓ-Y1ₘᵢₙ)/w1>0.1≥(X1ₘₐₓ-X1ₘᵢₙ)/c1, preferably (Y1ₘₐₓ-Y1ₘᵢₙ)/w1≥0.3>(X1ₘₐₓ-X1ₘᵢₙ)/c1;
b) a charging step in which all or part of said variable metal waste is introduced into a melting furnace, and is melted to obtain all or part of a liquid metal bath;
c) a measurement step wherein a composition of said liquid metal bath is continuously measured using a laser-induced plasma atomic emission spectrometry device also called LIBS, so as to continuously measure at least one content of said first element and a content of said second element in the liquid metal bath,
   the introduction of said variable metal waste during the charging step being interrupted when said content of said first element measured in the liquid metal bath reaches a value X1_{maxLIBS} such that 0.9*X1ₘₐₓ≤X1_{maxLIBS}≤X1ₘₐₓ, or when said content in said second element measured in the liquid metal bath reaches a value X2_{maxLIBS} such that 0.9*X2ₘₐₓ≤X2_{maxLIBS}≤X2ₘₐₓ,
d) an adjustment step implemented if the content of said first element measured in the liquid metal bath during the measurement step is less than the first lower target threshold X1ₘᵢₙ, in which an amount m2 of first defined metal waste is introduced into the melting furnace, said first defined metal waste being characterized by a first mean adjustment content v1 of said first element X1 lying between a lower adjustment threshold Z1ₘᵢₙ which is strictly greater than the first lower target threshold X1ₘᵢₙ, and an upper adjustment threshold Z1ₘₐₓ, and such that (Z1ₘₐₓ-Z1ₘᵢₙ)/v1<0.3;
e) a dilution step implemented if the content of said first element measured in the liquid metal bath during the measurement step is strictly greater than the first target upper threshold X1ₘₐₓ, or if the content of said second element measured in the liquid metal bath during the measurement step is strictly greater than the second target upper threshold X2ₘₐₓ in which an amount m4 of second defined metal waste is introduced into the melting furnace, said second defined metal waste being characterized by a mean dilution content v2 of said first element strictly lower than the first target upper threshold, and/or being characterized by a mean dilution content v3 of said second element strictly lower than the second target upper threshold (X2ₘₐₓ).

The arrangements described above and in particular steps a, b, c, d, and e making it possible to propose a preparation method for producing a liquid metal bath from variable metal waste and defined metal waste, without requiring a sorting step prior to the melting of the introduced metals. Thus, it is possible to obtain a liquid metal bath of precise composition from waste whose source is uncontrolled, coming partly from an external supplier, and whose composition is very variable.

The melting furnace may be a reverberation furnace, a rotary furnace, an induction furnace, a chimney furnace or a sidewell furnace.

Furthermore, continuous measurement of the composition by a LIBS method reduces the time required to obtain the composition of the liquid metal bath, which makes it possible to decrease the process time, and thus reduce the energy consumption associated with melting the metals.

Preferably, the charging step (b) in which all or part of said variable metal waste is introduced into a melting furnace is carried out continuously.

The preparation method may also have one or more of the following features, considered separately or in combination.

According to one embodiment, the dilution step (e) comprises introducing an amount m4 of second defined metal waste into the melting furnace, said second defined metal waste being characterized by a mean dilution content v2 pf said first element lying between 0 and the first lower threshold.

According to one embodiment, the dilution step (e) comprises introducing an amount m4 of second defined metal waste into the melting furnace, said second defined metal waste being characterized by a mean dilution content v3 of said second element strictly lower than the second target upper threshold.

According to one embodiment, the second content c2 is between 0 and the second target upper threshold X2ₘₐₓ. In general, the second content c2 does not correspond to a target value centered between 0 and X2ₘₐₓ, however, it is possible that the second content c2 is substantially equal to X2ₘₐₓ/2.

According to one embodiment, the first content c1 is substantially equal to (X1ₘₐₓ-X1ₘᵢₙ)/2.

According to one embodiment, before the charging step (b), the melting furnace comprises a liquid heel, said liquid heel comprising liquid metal from a previous process, and/or first defined metal waste, and/or second defined metal waste, said liquid heel then forming at least a part of the liquid metal bath.

According to one embodiment, the liquid heel is obtained by melting first defined metal waste and/or second defined metal waste.

According to one embodiment, (Y1ₘₐₓ-Y1ₘᵢₙ)/w1≥0.2 and (Z1ₘₐₓ-Z1ₘᵢₙ)/v1<0.2, preferably (Y1ₘₐₓ-Y1ₘᵢₙ)/w1≥0.3 and (Z1ₘₐₓ-Z1ₘᵢₙ)/v1<0.1.

According to one embodiment, Y1ₘᵢₙ≥X1ₘₐₓ.

According to one embodiment, Y1ₘᵢₙ<X1ₘᵢₙ.

According to one embodiment, 0.95*X1ₘₐₓ≤X1_{maxLIBS}≤0.99*X1ₘₐₓ or 0.95*X2ₘₐₓ≤X2_{maxLIBS}≤0.99*X2ₘₐₓ.

According to one embodiment, the charging step (b) comprises a regulation step taking into account the dissolution kinetics of the elements X1 and/or X2 According to one embodiment, the adjustment step (d) is implemented before the charging step (b).

According to one embodiment, the charging step (b) is implemented before the dilution step (e).

According to one embodiment, the measurement step (c) is implemented at any time, as soon as a liquid metal bath is present in the melting furnace. For example, the measurement step (c) is implemented by measuring the composition of the heel, in particular when it is melted.

It is also possible for the measurement step (c) to be implemented several times during the preparation method. For example, the measurement step (c) may be interrupted when metal is introduced into the melting furnace during an adjustment step (d) and/or during a dilution step (e). The measurement step (c) can then be implemented again following this introduction.

According to one embodiment, the charging (b), adjusting (d), and dilution (e) steps are implemented in any order.

According to one embodiment, during the adjustment step, an amount m3 of mother alloys and additive metals containing said first element is introduced into the melting furnace.

According to one embodiment, the variable metal waste and/or the first defined metal waste, and/or the second defined metal waste are respectively variable aluminum waste, first defined aluminum waste and second defined aluminum waste.

According to one embodiment, the liquid metal bath is an aluminum alloy of the series 2XXX, 3XXX, 5XXX, 6XXX, 7XXX, 8XXX.

According to one embodiment, the variable metal waste is waste as defined by the Institute of Scrap Recycling Industries (ISRI) in the ISRI Scrap Specifications Circular 2022 and in the section Guidelines for Nonferrous Scrap: NF-2022.

According to one embodiment, the variable metal waste is waste as defined by the Institute of Scrap Recycling Industries (ISRI) in the ISRI Scrap Specifications Circular 2022 and in the section Guidelines for Nonferrous Scrap: NF-2022, and in at least one subheading chosen from Magnesium, Zinc, and Aluminum, Mixed Metals or Red Metals.

According to one embodiment, the variable metal waste is of the Twitch or Zorba type, as defined by the Institute of Scrap Recycling Industries (ISRI) in the ISRI Scrap Specifications Circular 2022 and in the section Guidelines for Nonferrous Scrap: NF-2022.

### DESCRIPTION OF THE FIGURES

Other aspects, aims, advantages and features of the invention will appear better upon reading the following detailed description of preferred embodiments thereof, given as a non-limiting example, and made with reference to the appended drawings, wherein:

FIG. 1 is a schematic view of an example of the compositions of the alloys used in the preparation method according to an embodiment of the invention.

FIG. 2 is a schematic view of certain steps of the preparation method according to an embodiment of the invention.

FIG. 3 is a schematic view of different contents of elements of interest of the example.

FIG. 4 is a schematic view of the step of measuring the preparation process by LIBS according to the example.

FIG. 5 is a schematic view of the regulation step during the charging step.

### DETAILED DESCRIPTION

In the figures and in the remainder of the description, the same references represent identical or similar elements. In addition, the various elements are not plotted to scale so as to favor clarity of the figures. Moreover, the different embodiments and variants are not exclusive of one another and could be combined together.

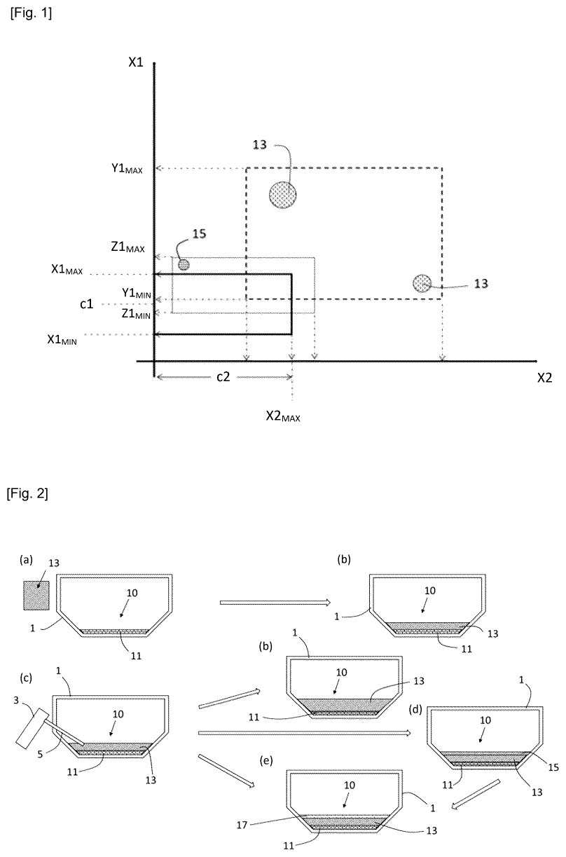

As illustrated in FIGS. 1 and 2, the invention relates to a method for preparing a liquid metal bath 10 of a metal alloy having a chemical composition comprising at least one first element X1 selected from Cu, Mg, Si, Zn, Li, Mn, Zr, Cr, Fe, Ti, V, Ca, Be, Sc, Cd, Pb, Na and Ni, present in said chemical composition at a first content c1 lying between a first lower target threshold X1ₘᵢₙ, and a first target upper threshold X1ₘₐₓ. In the particular case shown in FIG. 1, the first content c1 is substantially equal to (X1ₘₐₓ-X1ₘᵢₙ)/2. The metal alloy also comprises at least one second element X2 selected from Cu, Mg, Si, Zn, Li, Mn, Zr, Cr, Fe, Ti, V, Ca, Be, Sc, Cd, Pb, Na and Ni, present in said chemical composition at a second content c2, less than a second target upper threshold X2ₘₐₓ. In general, the first element X1 is strictly different from the second element X2.

In general, and as shown in FIG. 1, the second content c2 does not necessarily correspond to a target value centered between 0 and X2ₘₐₓ, however, it is possible for the second content c2 to be substantially equal to X2ₘₐₓ/2.

The preparation method first comprises a provision step (a) in which an amount m1 of variable metal waste 13 is provided, said amount m1 being counted in kilograms. As illustrated in FIG. 1, the variable metal waste 13 corresponds to metal waste characterized by a mean charge content w1 in said at least one first element X1 lying between a first lower initial threshold Y1ₘᵢₙ and a second upper initial threshold Y1ₘₐₓ, and such that Y1ₘₐₓ>X1ₘₐₓ, and such that (Y1ₘₐₓ-Y1ₘᵢₙ)/w1>0.1≥(X1ₘₐₓ-X1ₘᵢₙ)/c1. Thus, the interval in which the mean charge content w1 falls is greater than the range of the first targeted content c1. In an embodiment, (Y1ₘₐₓ-Y1ₘᵢₙ)/w1≥0.2, or even (Y1ₘₐₓ-Y1ₘᵢₙ)/w1≥0.3. Preferably (Y1ₘₐₓ-Y1ₘᵢₙ)/w1≥0.3>(X1ₘₐₓ-X1ₘᵢₙ)/c1.

Moreover, it is possible that the mean charge content w1 is very far from the first content c1, and in particular that Y1ₘᵢₙ≥X1ₘₐₓ, or that Y1ₘᵢₙ<X1ₘᵢₙ.

Unless otherwise stated, the definitions of the ISRI (Institute of Scrap Recycling Industries) “Scrap Specification Circular 2022” apply.

According to one embodiment, the variable metal waste 13 is waste as defined by the Institute of Scrap Recycling Industries (ISRI) in the ISRI Scrap Specifications Circular 2022 and in the section Guidelines for Nonferrous Scrap: NF-2022. According to the invention, when the waste is aluminum, the term “scrap” may also be used to comply with the EN12258-3 terminology; in this case, the term “aluminum waste” or “scrap” is equivalent in the context of the present invention. Preferably, variable metal waste 13 is waste as defined in at least one subsection of Guidelines for Nonferrous Scrap: NF-2022, chosen from Magnesium, Zinc, and Aluminum, Mixed Metals or Red Metals. Even more preferably, variable metal waste 13 belongs to the subheading Aluminum, and corresponds in particular to waste of the Twitch or Zorba type. In particular, twitch is defined as aluminum waste or scrap fragmented by automotive shredders. The twitch is derived from the wet or dry media separation device, the material is dry and does not contain more than a maximum of 1% free zinc, a maximum of 1% free magnesium and a maximum of 1% analytical iron. It contains no more than 2% in total of non-metallic materials, including no more than 1% of rubber and plastic. It is free of excessively oxidized materials, airbag cartridges, or any sealed or pressurized items.

The provision step (a) may also comprise providing first defined metal waste 15. The first defined metal waste 15 is also metal waste, but being characterized by a first mean adjustment content v1 of said first element X1 lying between a lower adjustment threshold Z1ₘᵢₙ which is strictly greater than the first target lower threshold X1ₘᵢₙ, and an upper adjustment threshold Z1ₘₐₓ, and such that (Z1ₘₐₓ-Z1ₘᵢₙ)/v1<0.3. It may also be provided that (Y1ₘₐₓ-Y1ₘᵢₙ)/w1≥0.2 and that (Z1ₘₐₓ-Z1ₘᵢₙ)/v1<0.2, and preferably that (Y1ₘₐₓ-Y1ₘᵢₙ)/w1≥0.3 and that (Z1ₘₐₓ-Z1ₘᵢₙ)/v1<0.1. According to one embodiment, the variable metal waste 13 and the defined metal waste 15 are such that (Y1ₘₐₓ-Y1ₘᵢₙ)/w1>(Z1ₘₐₓ-Z1ₘᵢₙ)/v1. This means that the composition of the variable metal waste 13 is less well known than that of the defined metal waste 15, according to the invention.

Furthermore, the provision step (a) may comprise providing mother alloys and additive metals containing said first element X1. Mother alloys mean alloys intended only to be added to a mixture to rectify the composition and/or structure of the raw cast product. Some mother alloys may contain more than 50% as the main additive element. In other words, the mother alloys correspond to alloys whose composition is known.

Finally, the provision step (a) may comprise the provision of second defined metal waste 17. These second defined metal waste 17 are characterized by a mean dilution content v2 in said first element X1 between 0 and the first lower target threshold X1ₘᵢₙ. It is therefore well understood that second defined metal waste 17 may comprise a pure metal, free of first element X1. Alternatively, it is possible for the second defined metal waste 17 to be characterized by a mean dilution content v2 in said first element X1 lying between 0 and the first target upper threshold X1ₘₐₓ. Said second defined metal waste may also be characterized by a mean dilution content v3 in said second element X2 strictly lower than the second target upper threshold X2 MAX.

In general, the variable metal waste 13 and/or the first defined metal waste 15, and/or the second defined metal waste 17 are respectively variable aluminum waste, first defined aluminum waste and second defined aluminum waste. Thus, it is possible to use the preparation method to produce a liquid metal bath 10 corresponding to an aluminum alloy of the series 2XXX, 3XXX, 5XXX, 6XXX, 7XXX, 8XXX. In particular, the series 2XXXX corresponds to aluminum alloys comprising copper in given proportions, and the series 6XXX corresponds to aluminum alloys comprising silicon and magnesium in given proportions.

The preparation method then comprises a charging step (b) in which all or part of said variable metal waste 13 is introduced, preferably continuously into a melting furnace 1, and is melted to obtain all or part of a liquid metal bath 10.

The melting oven 1 may be a reverberation oven, a rotary oven, an induction oven, preferably a crucible induction oven, a flue oven or a sidewell oven. The reverberation furnace is a type of furnace generally rectangular, covered with a refractory brick vault and a flue, which reflects (or reverberates) the heat produced in a location independent of the furnace where the heating system is located. Rotary furnaces consist of a cylindrical shell, with a substantially horizontal axis, which ends in two structures, one at each end. The burner is located at one end and the flue gas outlet is located at the other end, which usually passes through a heat recovery system to preheat the supply air before it is discharged through the flue. The entire interior of the furnace is lined with a refractory material. The fuel can be gas. The induction oven is preferably a crucible induction oven. The heater is an electromagnetic induction heater. A sidewell oven is an oven consisting of two chambers, a first chamber of which is used for charging, which communicates with the second chamber. All these types of furnace are well known to those skilled in the art.

It is possible that, before implementing the charging step (b), the melting furnace 1 already comprises a liquid heel 11 comprising liquid metal from for example a previous process, or first defined metal waste 15, and/or second defined metal waste 17.

To simplify the understanding of FIG. 2, the various introductions into the melting furnace 1 are represented by layers, however, it is well understood that once they are introduced, the materials are melted to form only a single liquid metal bath 10.

According to an alternative in which the liquid metal bath 10 is a liquid aluminum alloy bath, it can be provided that, during the loading step (b), the temperature prevailing in the melting chamber of the melting furnace 1 is placed at a value between 800° C. and 1000° C. In this way, it is possible to guarantee the melting of all the metal elements constituting the variable metal waste 13 and the other waste introduced into the melting chamber. It is also possible to burn any organic residues that would have been introduced, for example, with the variable metal waste 13.

Parallel or not to the charging step (b), a measurement step (c) is implemented, wherein a composition of said liquid metal bath 10 is continuously measured using a laser-induced plasma atomic emission spectrometry device 3 also called LIBS. This measurement step (c) is implemented so as to continuously measure at least one content of said first element X1 and a content of said second element X2 in the liquid metal bath 10. It is therefore well understood that this measurement step (c) can be implemented continuously throughout the preparation process, but also at isolated points in time, and this before, after or during the charging step (b). To implement the measurement step (c), the LIBS device 3 comprises a measurement probe 5 configured to incorporate argon bubbles in the liquid metal bath 10 so as to generate a plasma in said argon bubble; and to irradiate said plasma generated in said argon bubble by a laser to implement the laser-induced plasma atomic emission spectrometry method. Alternatively, it is possible to generate a plasma on the surface of the liquid metal bath 10 and irradiate said plasma by the laser to implement the laser-induced plasma atomic emission spectrometry method.

As illustrated in FIG. 2, during the measurement step (c), the measurement probe 5 can be introduced into the liquid metal bath 10 inclined at a measurement angle counted with respect to the surface of the liquid metal bath 10, said measurement angle being between 45° and 80°. Advantageously, the inclination of the measurement probe 5 makes it possible both to guarantee a good discharge of the argon bubbles out of the measurement probe 5, while making it possible to perform the measurement by laser-induced plasma atomic emission spectrometry.

Advantageously, it can be provided that the measurement step (c) further comprises a determination step in which a temperature sensor measures the temperature of the liquid metal bath 10. In this way, it is possible to know the temperature of the liquid metal bath 10. For example, the LIBS device 3 may comprise the temperature sensor, such that the measurements of the composition of the liquid metal bath 10 and of the temperature of the liquid metal bath 10 are implemented simultaneously.

The measurement step (c) makes it possible to determine conditions for interrupting the introduction of the variable metal waste 13 during the charging step (b). This is because such an introduction is interrupted when said content in said first element X1 measured in the liquid metal bath 10 reaches a value X1_{maxLIBS} such that 0.9*X1ₘₐₓ≤X1_{maxLIBS}≤X1ₘₐₓ or preferably 0.95*X1ₘₐₓ≤X1_{maxLIBS}≤0.99*X1ₘₐₓ, or when said content of said second element X2 measured in the liquid metal bath 10 reaches a value X2_{maxLIBS} such that 0.9*X2ₘₐₓ≤X2_{maxLIBS}X2ₘₐₓ or preferably 0.95*X2ₘₐₓ≤X2_{maxLIBS}≤0.99*X2ₘₐₓ. Preferably, it may be provided that the introduction of waste is interrupted when X1_{maxLIBS}=p*X1ₘₐₓ and/or X2_{maxLIBS}=p*X2ₘₐₓ, with p being able to take a value from 0.90 or 0.91 or 0.92 or 0.93 or 0.94 or 0.95 or 0.96 or 0.97 or 0.98 or 0.99 or 1.

According to a preferred embodiment, the charging step (b) comprises a regulation step taking into account the dissolution kinetics of the elements X1 and/or X2. The regulation based on the dissolution kinetics of the elements X1 and/or X2 is possible thanks to the continuous LIBS measurement of the content of the elements X1 and X2 in the liquid metal. It is possible to regulate the amount of variable metal waste to be added without exceeding the value X1_{maxLIBS} and/or X2_{maxLIBS} from the composition measured by LIBS of the element X1 and/or X2 at a time t. It is in fact possible to predict the dissolution of the element X1 or X2 by taking into account the derivatives of the composition curve measured as a function of time. A principle of the regulation step is illustrated in FIG. 5.

The dissolution kinetics depends not only on the element considered, but also on the morphology of the variable metal waste (size, geometry) considered, the temperature of the liquid metal, etc. Each dissolution kinetics is characterized by a function f linking the X1 (or X2) content as a function of time and the amount of variable metal waste added. The benefit of the LIBS is the possibility of continuously measuring the composition. It is thus possible, for a given type of variable metal waste and given melting conditions, to know at any time the change in the composition according to the quantity added. The advantage of this regulation step is to be able to avoid the dilution step (e).

The preparation method also comprises an adjustment step (d) implemented if the content of said first element X1 measured in the liquid metal bath 10 during the measurement step (c) is less than the first target lower threshold X1ₘᵢₙ. In this case, a quantity m2 of first defined metal waste 15 is introduced into the melting furnace 1. During this step, it is also possible for an amount m3 of mother alloys and additive metals containing said first element X1 to be introduced into the melting furnace 1, said amounts m2 and m3 being counted in kilograms. In this way, it is possible to adjust the composition of the liquid metal bath 10 from waste whose quantity is more precisely known. As specified previously, the adjustment step (d) can be implemented before the loading step b), for example to form the heel 11.

The preparation method may also comprise a dilution step (e) implemented if the content of said first element X1 measured in the liquid metal bath 10 during the measurement step (c) is strictly greater than the first target upper threshold X1ₘₐₓ, or if the content of said second element X2 measured in the liquid metal bath 10 during the measurement step (c) is strictly greater than the second target upper threshold X2ₘₐₓ. In this case, a quantity m4 of second defined metal waste 17 is introduced into the melting furnace 1, said quantity m4 being counted in kilograms. In general, the filling step (b) is implemented before the dilution step (e).

The arrangements described above and in particular the steps (a), (b), (c), (d), and (e) making it possible to propose a preparation method for producing a liquid metal bath 10 from variable and defined metal waste 13, without requiring a sorting step prior to the melting of the introduced metals. Thus, it is possible to obtain a liquid metal bath 10 of precise composition from waste partly from an external supplier, and the composition of which is very variable.

Furthermore, the continuous measurement of the composition by a LIBS method makes it possible to reduce the time necessary to obtain the composition of the liquid metal bath 10, which makes it possible to decrease the duration of the process, and therefore to reduce the energy consumption associated with the melting of the metals.

Following the preparation of the liquid metal bath 10, the preparation method may comprise a cleaning step in which a stirring device is introduced into the liquid metal bath 10 transferred into a holding chamber (not shown), to stir said liquid metal bath 10 so as to extract any gas contained in the liquid metal bath 10. In this way, it is possible to prepare a liquid metal bath 10 ready to be cast.

This cleaning step may further comprise the addition of a grain refining agent, in particular titanium, said grain refining agent being configured to enable germination of solid aluminum alloy grains. Advantageously, the addition of a grain refining agent makes it possible to obtain a better quality solid aluminum alloy after casting the liquid aluminum bath.

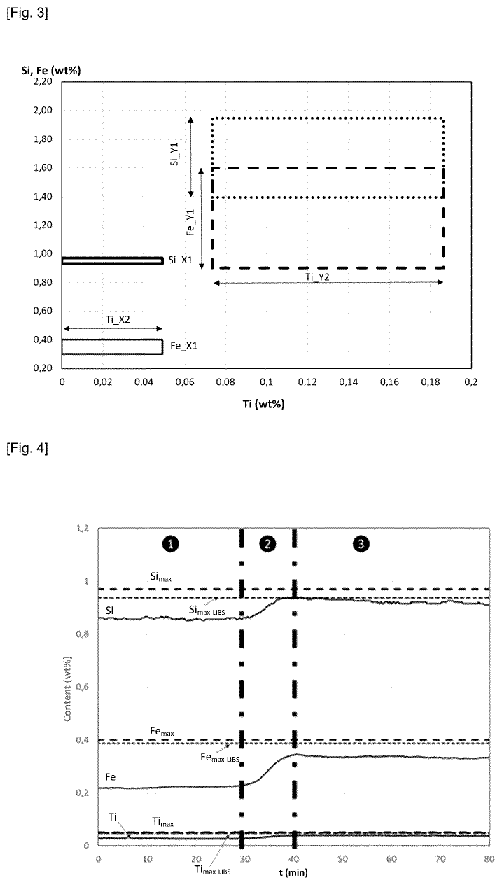

The preparation method and its implementation can be better understood based on the example given below with reference to FIGS. 3 to 5, given by way of non-limiting example.

### Example Implementation

For this example, the preparation method aims to prepare a liquid metal bath 10 of an aluminum alloy having a chemical composition comprising silicon Si, and iron Fe as first element X1. A c1si Si content equal to 0.95 wt % is aimed for. As shown in Table 1, the Si target composition, c1si, may consist of 0.93 wt % corresponding to X1_{MIN Si} to 0.97 wt % corresponding to X1_{MAX Si}, An Fe c1fe content equal to 0.35 wt % is also aimed at. As shown in Table 1, the c1fe target composition consists of 0.30 wt % corresponding to X1_{MIN FE} to 0.40 wt % corresponding to X1_{MAX FE}, The contents are expressed as a weight percentage, also noted as wt % or weight %. Thus, and as illustrated in Table 1, it is possible to extract the value of the ratios (Xₘₐₓᵢ-X1ₘᵢₙᵢ)/c1i. The target composition may also comprise a second element X2 corresponding to titanium Ti. Titanium may be present in said chemical composition at a second content c2ti, less than a second target upper threshold X2ₘₐₓ which is equal to 0.05 wt % in this example (Table 2).

TABLE 1
Target composition (by weight %) of
the liquid metal bath to be prepared

| | | X1_{min i} | C1i | X1_{max i} | (X1ₘₐₓ − X1ₘᵢₙ)/C1i | X2max |
|---|---|---|---|---|---|---|
| X1si | Si | 0.93 | 0.95 | 0.97 | 0.04 | |
| X1fe | Fe | 0.30 | 0.35 | 0.40 | 0.29 | |
| X2 | Ti | | | | | 0.05 |

Before proceeding with the charging step (b), a measurement step (c) was implemented in order to measure the composition of the liquid heel (11) contained in the melting oven 1, this liquid heel 11 consists of liquid metal from a previous process.

The measurement thus carried out makes it possible to obtain the composition of the liquid metal bath shown in Table 2. The liquid heel contains Si, Fe and Ti, the rest being aluminum.

TABLE 2
Composition of the liquid heel (by weight %)

| | Si | Fe | Ti | Al |
|---|---|---|---|---|
| Measured | 0.82 | 0.21 | 0.03 | remainder |

A step of providing (a) variable metal waste 13 is then implemented. The variable metal waste 13 is characterized by the composition described in Table 3 concerning the elements Si, Fe and Ti respectively (Y1si, Y1fe, Y2). It is possible to extract the value of the ratios (Yₘₐₓᵢ-Yₘᵢₙi/wᵢ).

TABLE 3
Composition of variable metal waste (by weight %)

| | | Y1_{min i} | Wᵢ | Y1_{max i} | Y1max − X1max)/W i |
|---|---|---|---|---|---|
| Y1si | Si | 1.39 | 1.67 | 1.95 | 0.33 |
| Y1fe | Fe | 0.90 | 1.25 | 1.60 | 0.56 |
| Y2 | Ti | 0.07 | 0.13 | 0.19 | 0.87 |

The graphical comparison between the variability of the composition of the metal (Y1si, Y1fe, Y2) and that of the target (X1si, X1fe, X2) is shown in FIG. 3.

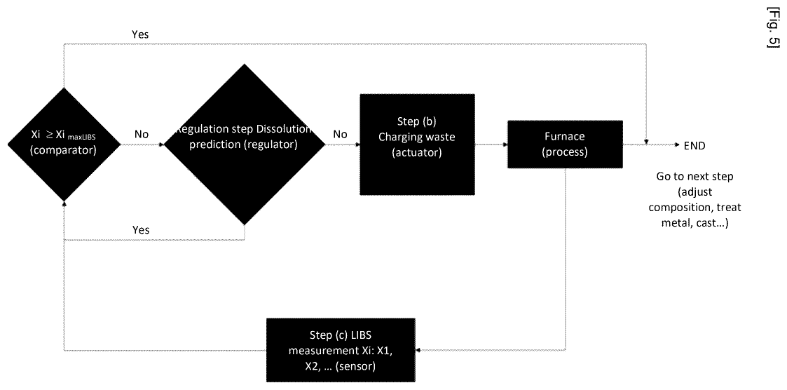

To manufacture a liquid metal bath 10 having a composition that tends towards the target composition (X1si, X1fe, X2), the measurement step (c) is implemented continuously and in real time using a LIBS sensor. FIG. 4 shows the composition of the liquid metal bath in each of the elements Si, Fe and Ti as a function of time during the measurement step (c).

As can be seen in FIG. 4, the preparation method comprises the following steps:
1. Melting and stabilization of the liquid-heel composition;
2. a charging step (b) comprising adding variable metal waste 13;
3. reaching the target composition by interrupting the charging step (b).

FIG. 4 illustrates in particular the change in the composition of the elements Si, Fe, and Ti measured during the measurement step (c) over time.

As can be seen in FIG. 4, the charging step (b) is interrupted when the content of at least one of the elements (Si, Fe or Ti) has reached the limit 0.97*X1ₘₐₓ. Here, it is the Si content that reached the value of 0.94 wt % which is equal to 0.97*X_{max Si} in the vicinity of a time of 40 min.

In this case, at the end of the interruption, the final composition obtained being within the intervals defined in Table 1, it is therefore not planned to implement an adjustment step (d), nor a dilution step (e).

1. A method for preparing a liquid metal bath (10) of a metal alloy having a chemical composition comprising at least one first element (X1) selected from Cu, Mg, Si, Zn, Li, Mn, Zr, Cr, Fe, Ti, V, Ca, Be, Sc, Cd, Pb and Na present in said chemical composition at a first content c1 lying between a first lower target threshold (X1ₘᵢₙ), and a first target upper threshold (X1ₘₐₓ); and at least one second element (X2) selected from Cu, Mg, Si, Zn, Li, Mn, Zr, Cr, Fe, Ti, V, Ca, Be, Sc, Cd, Pb and Na present in said chemical composition at a second content c2, lower than a second target upper threshold (X2ₘₐₓ), the method comprising:
a provisioning step (a) in which an amount m1 of variable metal waste (13) is provided, said variable metal waste (13) being characterized by a mean charge content w1 of said at least one first element (X1) lying between a first lower initial threshold (Y1ₘᵢₙ) and a second upper initial threshold (Y1ₘₐₓ), wherein Y1ₘₐₓ>X1ₘₐₓ and (Y1ₘₐₓ-Y1ₘᵢₙ)/w1>0.1≥(X1ₘₐₓ-X1ₘᵢₙ)/c1;
a charging step (b) in which all or part of said variable metal waste is introduced into a melting furnace (1), and is melted to obtain all or part of a liquid metal bath (10);
a measurement step (c) wherein a composition of said liquid metal bath (10) is continuously measured using a laser-induced plasma atomic emission spectrometry device (3) also called LIBS, so as to continuously measure at least one content of said first element (X1) and a content of said second element (X2) in the liquid metal bath (10),
introducing said variable metal waste (13) during the loading step (b) being interrupted when said content of said first element (X1) measured in the liquid metal bath (10) reaches a value X1_{maxLIBS}, wherein 0.9*X1ₘₐₓ≤X1_{maxLIBS}≤X1ₘₐₓ, or when said content of said second element (X2) measured in the liquid metal bath (10) reaches a value of X2_{maxLIBS}, wherein 0.9*X2ₘₐₓ≤X2_{maxLIBS}≤X2ₘₐₓ;
an adjustment step (d) implemented if the content of said first element (X1) measured in the liquid metal bath (10) during the measurement step (c) is less than the first target lower threshold (X1ₘᵢₙ), in which an amount m2 of first defined metal waste (15) is introduced into the melting furnace (1), said first defined metal waste (15) being characterized by a first mean adjustment content v1 of said first element (X1) lying between a lower adjustment threshold (Z1ₘᵢₙ) which is strictly greater than the first lower target threshold (X1ₘᵢₙ), and an upper adjustment threshold (Z1ₘₐₓ), wherein (Z1ₘₐₓ-Z1ₘᵢₙ)/v1<0.3;
ea dilution step (e) implemented if the content of said first element (X1) measured in the liquid metal bath (10) during the measurement step (c) or (d) is strictly greater than the first target upper threshold (X1ₘₐₓ), or if the content of said second element (X2) measured in the liquid metal bath (10) during the measurement step (c) is strictly greater than the second target upper threshold (X2ₘₐₓ) wherein an amount m4 of defined primary aluminum or second metal waste (17) is introduced into the melting furnace (1), said second defined metal waste (17) being characterized by a mean dilution content v2 in said first element (X1) strictly lower than the first target upper threshold (X1ₘₐₓ), and/or being characterized by a mean dilution content v3 of said second element (X2) strictly below the second target upper threshold (X2ₘₐₓ).

2. The method according to claim 1, wherein, before the filling step (b), the melting furnace (1) comprises a liquid heel (11), said liquid heel (11) comprising liquid metal from a previous process, first defined metal waste (15), and/or second defined metal waste (17), said liquid heel then forming at least a part of the liquid metal bath.

3. The method according to claim 1, wherein (Y1ₘₐₓ-Y1ₘᵢₙ)/w1≥0.2 and wherein (Z1ₘₐₓ-Z1ₘᵢₙ)/v1<0.2, and preferably wherein (Y1ₘₐₓ-Y1ₘᵢₙ)/w1≥0.3 and wherein (Z1ₘₐₓ-Z1ₘᵢₙ)/v1<0.1.

4. The method according to claim 2, wherein Y1ₘᵢₙ≥X1ₘₐₓ.

5. The method according to claim 1, wherein Y1ₘᵢₙ<X1ₘᵢₙ.

6. The method according to claim 1, wherein 0.95*X1ₘₐₓ≤X1_{maxLIBS}≤0.99*X1ₘₐₓ or 0.95*X2ₘₐₓ≤X2_{maxLIBS}≤0.99*X2ₘₐₓ.

7. The method according to claim 1, wherein the filling step (b) comprises a regulation step taking into account the dissolution kinetics of the elements X1 and/or X2.

8. The method according to claim 1, wherein the adjustment step (d) is implemented before the charging step (b).

9. The method according to claim 1, wherein the charging step (b) is implemented before the dilution step (e).

10. The method according to claim 1, wherein the variable metal waste (13) and/or the first defined metal waste (15), and/or the second defined metal waste (17), are respectively variable aluminum waste, first defined aluminum waste and second defined aluminum waste, and wherein the liquid metal bath (10) is an aluminum alloy of the series 2XXX, 3XXX, 5XXX, 6XXX, 7XXX, 8XXX.

11. The method according to claim 10, wherein the variable metal waste (13) is of the Twitch or Zorba type, as defined by the Institute of Scrap Recycling Industries (ISRI) in the ISRI Scrap Specifications Circular 2022 and in the Guidelines for Nonferrous Scrap: NF-2022.
